(19) United States
(12) Patent Application Publication
(10) Pub. No.: US 2023/0389820 A1
(43) Pub. Date: Dec. 7, 2023

(54) DEVICE FOR MEASURING AN INNER WIDTH OF A POCKET ARRANGED IN A SURGICAL PLANE DEEP TO THE SKIN OR UNDERLYING TISSUE

(71) Applicant: FERIDA SOLUTIONS AB, Linköping (SE)

(72) Inventors: Johan Thorfinn, Linkoping (SE); Per Heden, Stockholm (SE)

(21) Appl. No.: 18/026,490

(22) PCT Filed: Sep. 13, 2021
(86) PCT No.: PCT/SE2021/050876

§ 371 Date: Mar. 15, 2023

(30) Foreign Application Priority Data
Sep. 15, 2020 (SE) 2051076-4

(51) Int. Cl. A61B 5/107; A61B 90/00

(52) U.S. Cl. CPC A61B 5/1076; A61B 90/06; A61B 2090/061

(57) ABSTRACT

Device (1) for measuring an inner width (D) of a pocket (11) arranged in a surgical plane deep to the skin or underlying tissue. The device comprises two legs (2, 3), each having a first leg portion (2a, 3a) and a second leg portion (2b, 3b) fixed to each other at a joint connection (2c, 3c). The legs (2, 3) being connected to each other at the joint connections (2c, 3c), such that the legs (2, 3) are pivotable relative each other in a plane (XY), such that when end portions (2a2, 3a2) of the two first leg portions (2a, 3a) are moved towards each other in the plane (XY), end portions (2b2, 3b2) of the second leg portions (2b, 3b) are caused to move apart from each other in the plane (XY). A distance reader (6) gives a distance (B) between abutment surfaces of the end portions (2b2, 3b2) of the second leg portions (2b, 3b) in the plane (XY). An angle (D2, D3) between the first and second leg portions (2a, 3a; 2b, 3b) of each leg (2, 3) at the joint connection (2c, 3c) is 90 to 150 degrees.

### TECHNICAL FIELD

The present document relates to a device for measuring an inner width of a pocket arranged in a surgical plane deep to the skin or underlying tissue.

### BACKGROUND ART

Breast implants on the market are supplied in different sizes and generally have a diameter of about 10-15 cm. A difference in diameter between two consecutive breast implant sizes normally differ as little as with 1-5 mm. When inserting a breast implant, it is important that the pocket created for the implant has an exact size, i.e. that there is a close fitting between the pocket and the implant. If the pocket is too big, there is a risk that the implant will move around in the pocket after insertion, especially if the implant is of the type having a smooth outer surface, and end up in a non-optimal position once healed.

The pocket for the implant is created behind or in front of the breast muscle (pectoralis major) by forming an incision in the skin at or near the lower border of the breast (inframammary fold) when the patient is lying on the operating table in the supine position. Such an incision may for example be about 2-5 cm long. When the surgeon creates the pocket for the implant it is difficult to get a full overview of the pocket through the small incision, and the exact width (diameter) of the pocket can thus only be estimated.

Being able to measure the width of the pocket at its widest part, located at a distance from the incision, for example 5-8 cm from the incision, and parallel with the incision, in an exact manner, would be desirable for providing pockets with a close fitting with the implant of choice. Today there is no known method or instrument for measuring such pocket width and the result of the implantation is merely dependent on the surgeon's the estimation of the width.

### SUMMARY OF THE INVENTION

It is an object of the present disclosure to provide a device for measuring the inner width of a pocket arranged in a surgical plane deep to the skin or underlying tissue.

The invention is defined by the appended independent patent claims. Non-limiting embodiments emerge from the dependent patent claims, the appended drawings and the following description.

According to a first aspect there is provided a device for measuring an inner width of a pocket arranged in a surgical plane deep to the skin or underlying tissue of a subject. The device comprises two legs, wherein each leg has a first leg portion and a second leg portion, the first and second leg portions of a leg being fixed to each other at a joint connection, the second leg portions being provided with a respective abutment surface at a respective end portion thereof for abutment against an inner wall of the pocket. The two legs being connected or connectable to each other at the joint connections, such that a main direction of extension of the two connected legs is substantially in one plane, wherein the connected legs are pivotable relative each other in the plane about an axis perpendicular to the plane, in such a way that when the end portions of the two first leg portions are moved towards each other in the plane, the end portions of the two second leg portions are caused to move apart from each other in the plane, and when the end portions of the two first leg portions are moved apart from each other in the plane, the end portions of the two second leg portions are caused to move towards each other in the plane. A distance reader is arranged to give a current distance between the abutment surfaces of the two second end portions of the second leg portions of the legs in the plane. An angle between the first leg portion and the second leg portion of each respective leg at the joint connection is 90 to 150 degrees.

Using the present device, the inner width of a pocket created for example behind or in front of the breast muscle (pectoralis major) for receiving a breast implant can be measured. The current (closest) distance between the abutment surfaces corresponds to an inner width of the pocket at the point of measurement. The device is not limited to measuring inner widths of pockets behind or in front of the breast muscle, but may be used to measure the inner width of any pocket arranged in any surgical plane deep to the skin or underlying tissue, such as a subcutaneous plane, a subglandular plane, a subfascial plane, a submuscular plane or in dual planes.

That a main direction of extension of the two connected legs is substantially in one plane, is here meant that there might be a smaller deviation from the plane with a portion of one or both of the legs, such as 0-5 mm, or 0-10 mm, or 5-10 mm.

The angle between the first and second leg portion at the joint connection may be 90-150, or 90-140, or 90-130, or 90-120, or 90-110, or 90-100, or 100-130, or 110-150, or 110-130, or 120-130, or 100-120 degrees. In one embodiment, both legs have approximately the same angle. In another embodiment a difference in angles between the first and second legs may be 10-30 degrees. The angle at the joint connection is here an angle measured between the first and second leg portions close to the connection point and no more than 1-2 cm from the connection point. When the two legs are connected to each other, the two second leg portions may have a major direction of extension in the same XY plane, while there may be a smaller difference between the second leg portions (at least at the joint connections) in a direction (Z) perpendicular to the plane.

When the end portions of the second leg portions have been moved towards each other, and as a result the second leg portions have been moved towards each other, the second leg portions may be inserted through a skin incision and into the pocket. The ends of the second leg portions may be inserted through the incision such that a major direction of extension of the two second leg portions is substantially perpendicular to the direction of extension of the incision. Alternatively, the ends of the two second leg portions may be inserted through the incision at an angle to the incision to facilitate insertion in some instances. Once inside the pocket, with the joint connection at approximately the same level as the skin incision, the end portions of the second leg portions may be moved apart from each other by moving the end portions of the first leg portions towards each other, such that the abutment surfaces are moved towards the walls of the pocket and stopped when abutting the inner walls. At this position of the device, an inner width of the pocket can then be measured as a closest distance between the two abutment surfaces.

Due to the angle of 90-150 degrees between the first and second leg portions, it is possible to insert the second leg portions through a skin incision being much smaller than the width of the pocket that is to be measured.

The legs of the device may be inserted through an incision in a connected state. The legs may be fixedly connected to each other. Alternatively, the two legs may be removably/detachably connectable to each other.

When the end portions of the first leg portions of the legs are at a maximum distance from each other in the plane, the end portions of the second leg portions of the legs are at a closest distance to each other and the second leg portions extend in substantially the same direction in the plane along a centre line extending through the joint connections in the plane, such that a cross-sectional area of the two second leg portions, including any space in between the second leg portions, taken perpendicular to the centre line may be at most 4 cm².

A cross-sectional area may be at most 4 cm², 3 cm², or 2 cm².

When at a closest distance to each other the second leg portions may be inserted through a skin incision of 1-5 cm, 1-4, 1-3, 1-2, 2-5, 3-5, or 2-4 cm.

That the second leg portions extend in substantially the same direction in the plane along a centre line extending through the joint connections in the plane is here meant that here might be a smaller deviation from the centre line with a portion of one or both legs leg, such as 0-5 mm, or 0-10 mm, or 5-10 mm, or 5-15 mm, or 10-15 mm.

The second leg portion may have a length of 4-12 cm.

Normally, the widest part of a pocket is located about 5-8 cm from the incision site. The device and the length of the second leg portions may be chosen depending on the type of surgery/measurement to be performed and to the incision/pocket size.

The second leg portion may have a length of 4-12, 4-6, 4-8, 4-10, 6-8, 8-10, or 6-cm.

The length of the first leg portions may be any suitable to the specific application. The length of the first leg portions does not affect the maximum width of the pocket than can be measured.

A largest closest distance between the abutment surfaces at the end portions of the second leg portions of the legs in the plane may be 20 cm.

A largest closest distance may be 20 cm, or 18 cm, or 16 cm, or 14 cm, or 12 cm, or 10 cm. The largest distance being dependent on the length of the second leg portions and the angle between the first and second leg portions. The largest distance obtainable between the two abutment surfaces determines how wide a pocket the device can measure.

At least a portion of at least one of the second leg portions may be arched such that, when the end portions of the two first leg portions of the legs are at a maximum distance from each other in the plane and the end portions of the second leg portions of the legs are at a closest distance to each other and the second leg portions extend in substantially the same direction along a centre line extending through the joint connections in the plane, a radius of curvature of the arched portion of a second leg portion of a leg is located in or extends through an area defined in the plane between the first and second leg portions of that leg.

The centre of curvature may be located in the area defined between the first and second leg portions in the plane, or outside of this area, depending on the degree of curvature of the second leg portion. At least a portion of the second leg portion may be arched. Alternatively, the whole length of the second leg portion may be arched. The second leg portion may be provided with one arched portion or two of more arched portions. One of the legs may be provided with a second leg having at least one arched portion. Alternatively, both legs may be provided with second leg portions having one or more arched portions.

Arched such second leg portions may make the movement of the end portions of the second leg portions away from each other in the pocket a more gentle movement against the inner walls of the pocket compared to a device having straight second leg portions. Further, the end portions may follow the anatomy of the chest in a more precise way.

By making at least a portion of at least one of the second leg portions arched/curved, or the whole second leg portion arched, it may be possible to increase the widest width of the pocket that can be measured with the device

At least a portion of at least one of the second leg portions may be arched such that when the end portions of the two second leg portions of the legs are at a closest distance to each other in the plane, at least a portion of a second leg portion cross at least a portion of the other second leg portion and the centre line in the plane.

The arched portion may be arched such that a radius of curvature of the curved portion is 8-15 cm.

The radius of curvature may be 8-15 cm, or 10-15, or 12-15, or 8-12 or 10-12 cm.

At least a portion of one of the second leg portions may be arched such that there is a curvature in a direction perpendicular to the plane.

Such a curvature may make the second leg portions better follow the shape of the thorax from the sternum to the lateral aspect of the thorax to the approximate level of the midclavicular line when in use.

When the end portions of the first leg portions of the legs are at a maximum distance from each other in the plane, an angle between the first leg portions of the two legs at the joint connection may be 130-180 degrees, and an angle between the second leg portions of the two legs at the joint connection may be 0-10 degrees.

In one embodiment, the angle between the two second leg portions at the joint connection may be 0-5 degrees.

The distance reader may be arranged such that a current (closest) distance between the abutment surfaces of the end portions of the second leg portions of the legs may be derivable from a current distance or angle between the first leg portions of the legs at a position between the joint connection and the end portions of the first leg portions of the legs.

The distance reader may be constituted by a protractor arranged at a position between the joint connection and the end portions of the first leg portions of the legs, and wherein the position of one of the first leg portions at/above the protractor may indicate the distance or angle between the first leg portions of the legs at the position of the protractor, this distance or angle corresponding to the current (closest) distance between the abutment surfaces of the end portions of the second leg portions of the legs in the plane.

The distance reader may comprise an elongated member rotatably connected to one of the two first leg portions at a first end of the elongated member, an indicator fixedly connected to the other of the two first leg portions, wherein the elongated member and the indicator may be arranged such that the indicator is slidable along the elongated member as the end portions of the first leg portions of the first and second legs are moved towards and apart from each other, wherein the elongated member may be provided with a measurement surface with a scale such that the position of the indicator along the scale correlates with a current distance between the two abutment surfaces of the end portions of the second leg portions of the legs.

The elongated member may be provided with an elongated slot extending at least along a portion of the length of the elongated member, along which slot the indicator is allowed to move as the end portions of the first leg portions of the first and second legs are moved towards and apart from each other.

The elongated member may be connected to one of the two first leg portions at a distance from the connection joint and the indicator may be fixed to the other of the two first leg portions at substantially the same distance from the connection joint.

The elongated member may be essentially straight.

That the elongated member is connected to the two leg portions at substantially the same distance from the connection joint is here meant that the distance of the connection point of the elongated member from the connection joint at on leg may differ with 0-5 mm, 0-mm, or 5-10 mm from the distance of the connection point of the elongated member from the connection joint at the other leg.

The elongated member may be rotatable connected to a first leg portion by means of for example a screw, a rivet, a magnet, or a snap connection, which rotates when the end portions of the first leg portions are moved towards each other and apart from each other. Further, the design of the distance reader keeps the elongated member constantly parallel to the skin incision during use of the device.

The two legs may be connected at the joint connection by a mechanical connection, a form-fit connection or a magnetic connection.

Examples connections are a rivet, a screw, a magnet, or a snap connection.

The device may further comprise cutting means for use when forming the pocket.

The formation of the pocket may take place before and/or during the measurement of the inner width of the pocket. Such a device, hence, aids a surgeon not only with measuring a pocket width, but also with creating a pocket with exact dimensions, thereby improving the result of the implantation.

The device may further comprise a light source arranged to provide light into the pocket.

Light may be provided in the surgical field inside the pocket to give the surgeon a better view of the pocket.

According to a second aspect there is provided a method of measuring an inner width of a pocket arranged in a surgical plane deep to the skin or underlying tissue using the device described above. The method comprising to insert the second leg portions of the legs through a skin incision into the pocket, causing the end portions of the two second leg portions of the device to move apart from each other in the plane such that the respective abutment surfaces of the end portions of the second leg portions abut against an inner wall of the pocket, by means of the distance reader measure a current closest distance between the abutment surfaces of the end portions of the second leg portions of the legs, the measured distance corresponding to the inner width of the pocket at the position of the end portions of the second leg portions of the legs of the device in the pocket.

When the device comprises cutting means for use when forming the pocket, the method may further comprise to form the pocket before/while measuring the width thereof.

### BRIEF DESCRIPTION OF THE DRAWINGS

FIG. 1 shows a device for measuring an inner width of a pocket arranged in a surgical plane deep to the skin or underlying tissue for a breast implant where the device is inserted through an incision located at a distance from the point of measurement in the pocket.

FIG. 2 shows a device for measuring an inner width of a pocket, wherein the two legs are pivotably connected to each other such that when the two first ends of the legs are moved apart from each other in the plane, the two second ends of the legs are caused to move apart from each other in the plane.

FIG. 3 shows a device for measuring an inner width of a pocket, wherein the two legs are pivotably connected to each other such that when the two first ends of the legs are moved apart from each other in the plane, the two second ends of the legs are caused to move towards each other in the plane. The device is further shown with a light source arranged to provide light into the pocket.

FIG. 4 shows an embodiment of a second end of the first and/or second leg of the device shown in FIG. 2 or 3 provided with an enlarged abutment surface.

FIG. 5 shows a device for measuring an inner width of a pocket, wherein the device has two legs and each leg has a first leg portion and a second leg portion fixed to each other at a joint connection. An angle between the first leg portion and the second leg portion of a leg at the joint connection is 90 to 150 degrees. The joint connections of the first and second legs are connected or connectable to each other, such that a main direction of extension of the two connected legs is substantially in one plane, and the connected legs are pivotable relative each other in the plane around an axis perpendicular to the plane. In FIG. 5, the device is shown with a maximum distance between the end portions of the first leg portions and a minimum distance between the end portions of the second leg portions.

FIG. 6 shows the same device as is shown in FIG. 5. In FIG. 6, the device is shown when the end portions of the first leg portions have moved away from a maximum distance from each other and the end portions of the second leg portions have moved away from a minimum distance from each other.

FIG. 7a shows an embodiment of a device for measuring an inner width of a pocket, wherein the device is provided with cutting means for forming the pocket before/while measuring the width thereof.

FIG. 7b shows a system comprising the device of FIG. 7a.

FIG. 8 shows a device for measuring an inner width of a pocket further provided with an arrangement for measuring a depth of the pocket.

### DETAILED DESCRIPTION

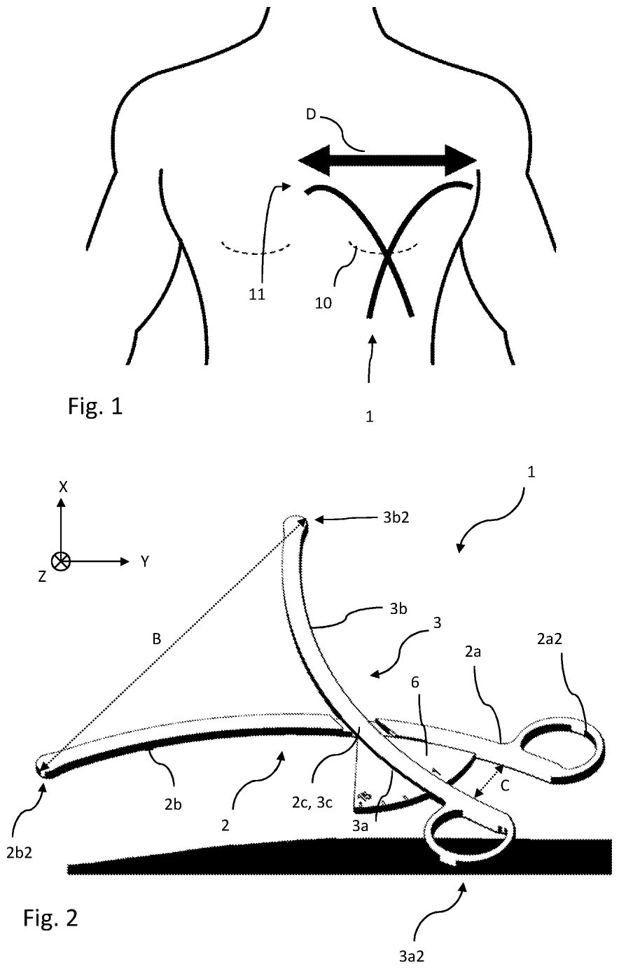

Breast implants on the market are supplied in different sizes and generally have a diameter of about 10-15 cm. A difference in diameter between two consecutive breast implant sizes normally differ with as little as with 1-5 mm. When inserting a breast implant, it is important that the pocket 11, see FIG. 1, created for the implant has an exact size, i.e. that there is a close fitting between the pocket 11 and the implant. If the pocket 11 is too big, there is a risk that the implant will move around in the pocket, especially if the implant is of the type having a smooth outer surface, and end up in a non-optimal position once healed. The pocket 11 for the implant is created behind or in front of the breast muscle (pectoralis major) by forming an incision 10 in the skin at or near the lower border of the breast (inframammary fold) when the patient is lying on the operating table in the supine position. Such an incision may for example be about 2-5 cm long. The creation of the pocket 11 is difficult by the fact that it is not possible for the surgeon to get a full overview of the pocket from the skin incision.

With the device 1 shown in FIGS. 2, 3, 5 and 6 it is possible to measure the inner width D of the pocket 11 at its widest part, which for example may be about 5-8 cm from the incision 10 and parallel with the incision, in an exact manner (see FIG. 1). Thereby, pockets 11 with a close fitting with the implant of choice can be formed.

The device 1, see FIGS. 2, 3, 5 and 6 comprises two legs 2, 3 wherein each leg has a first 2a, 3a and a second leg portion 2b, 3b. The second leg portions 2b, 3b of the legs are provided with a respective abutment surface at respective end portions 2b2, 3b2 thereof for abutment against an inner wall of the pocket 11. The first and second leg portions 2a, 3c; 2b, 3b of a leg 2, 3 are fixed to each other at a joint connection 2c, 3c. A leg may comprise two interconnected parts, a first leg portion and a second leg portion, or be a leg made in one piece. The leg portions may be extendable. The leg portion may be exchangeable to match the site of measurement.

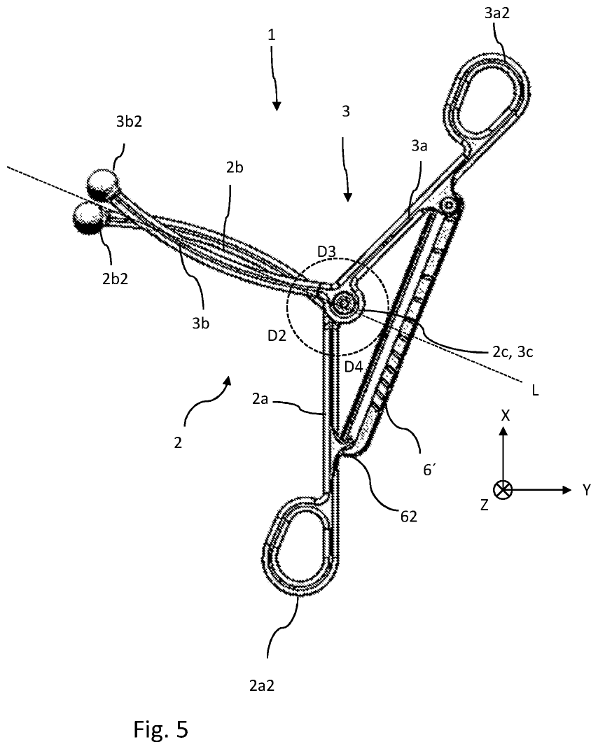

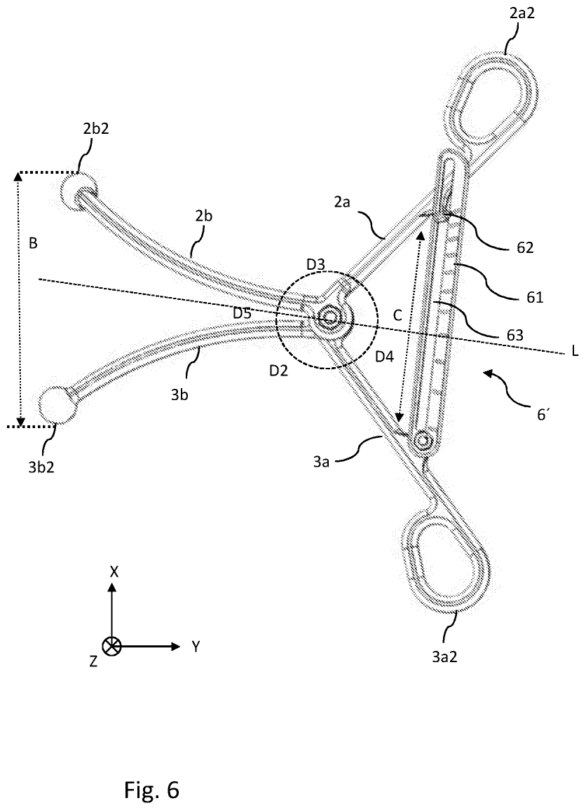

The abutment surface may be of any shape and size and may for example be formed as in FIG. 2, 3, 4 or 5. The abutment surface may in one embodiment be an enlarged portion at the end portions 2b2, 3b2 of the second leg portions 2b, 3b of the legs 2, 3. The abutment surface may be formed such as not to cause injury to the inner wall of the pocket when abutting against the inner wall. The end portions 2b2, 3b2 of the second leg portions 2b, 3b may for example be provided with balls or beads (see e.g. FIG. 5 or 6) on which the abutment surface is located.

The end portions 2a2, 3a2 of the first leg portions 2a, 3a of the legs 2, 3 may be provided with loops as illustrated in for example FIG. 2.

In FIGS. 2, 3, 5 and 6 are illustrated a device wherein the two legs 2, 3 are connected or connectable to each other at the joint connections 2c, 3c such that a main direction of extension of the two connected legs is in one plane XY. The connection is a pivotable connection and the legs 2, 3 are pivotable relative each other in the plane XY around an axis Z perpendicular to the plane XY.

The two legs 2, 3 may be non-releasably, pivotably connected to each other or may be releasably connectable to each other and may for example be connected through a mechanical, magnetic or form-fit connection. Examples of connections are a rivet, a screw, a magnet or a snap connection.

In the embodiment shown in FIG. 2 the two legs 2, 3 are pivotably connected to each other such that when the end portions 2a2, 3a2 of the two first leg portions 2a, 3a of the legs 2, 3 are moved apart from each other in the plane XY the end portions 2b2, 3b2 of the two second leg portions 2b, 3b of the legs 2, 3 are caused to move apart from each other in the plane XY.

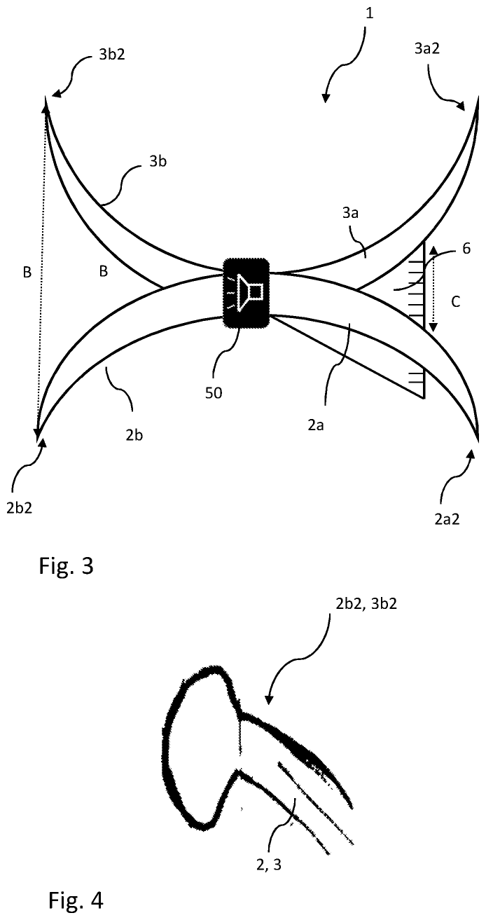

Alternatively, as is shown in FIGS. 3, 5 and 6, the two legs 2, 3 are pivotably connected to each other such that when the end portions 2a2, 3a2 of the two first leg portions 2a, 3a of the legs 2, 3 are moved apart from each other in the plane XY the end portions 2b2, 3b2 of two second leg portions 2b, 3b of the legs 2, 3 are caused to move towards each other in the plane XY, and when the end portions 2a2, 3a2 of the two first leg portions 2a, 3a are moved towards each other in the plane XY, the end portions 2b2, 3b2 of the second leg portions 2b, 3b are caused to move apart from each other in the plane XY.

As illustrated in FIGS. 5 and 6, an angle D2, D3 between the first leg portion 2a, 3a and the second leg portion 2b, 3b of the respective leg 2, 3 at the joint connection 2c, 3c may be 90 to 150 degrees.

The second leg portions 2b, 3b of the two legs 2, 3 may be substantially straight. Alternatively, as illustrated in FIGS. 1-3, the leg portions 2b, 3b may be arched in such a way that when the end portions 2a2, 3a2 of the first leg portions 2a, 3a of the legs 2, 3 are brought towards each other, the end portions 2b2, 3b2 of the second leg portions 2b, 3b of the legs 2, 3 are at a distance from each other in the plane XY.

At least a portion of at least one of the second leg portions may be arched such that, when the end portions 2a2, 3a2 of the two first leg portions 2a, 3a of the legs 2, 3 are at a maximum distance from each other in the plane XY and the end portions 2b2, 3b2 of the second leg portions 2b, 3b of the legs 2, 3 are at a closest distance to each other and the second leg portions 2b, 2b extend in substantially the same direction along a centre line L extending through the joint connections in the plane XY, a radius of curvature of the arched portion of the second leg portion may be located in or extend through an area defined in the plane XY between the first 2a, 3a and second 2b, 3b leg portions of a leg 2, 3. See FIG. 5.

As illustrated in FIG. 5, at least a portion of at least one of the second leg portions may be arched such that when the end portions 2b2, 3b2 of the two second leg portions 2b, 3b of the legs 2, 3 are at a closest distance to each other in the plane XY, at least a portion of a second leg portion 2b, 3b cross the other second leg portion 2b, 3b and the centre line L in the plane XY.

The arched portion may be arched such that a radius of curvature is 8-15 cm.

At least a portion of one of the second leg portions may be arched such that there is a curvature in a direction Z perpendicular to the plane XY.

In one embodiment, when the end portions 2a2, 3a2 of the first leg portions 2a, 3a of the legs 2, 3 are at a maximum distance from each other in the plane XY, an angle D4 between the first leg portions 2a, 3a of the two legs 2, 3 at the joint connection 2c, 3c is 130-180 degrees, and an angle D5 between the second leg portions 2b, 3b of the two legs 2, 3 at the joint connection is 0-10 degrees (see FIG. 6)

The legs 2, 3 of the device 1 may have a length of 4-12 cm, such as at least 5 cm. An average thickness of a leg may be for example 5-10 mm. At least one of the legs 2, 3 may be provided with a material thinning at the area of connection of the two legs 2, 3 such as to facilitate the pivotable movement of the two legs 2, 3 relative each other.

When the end portions 2a2, 3a2 of the first leg portions 2a, 3a of the legs 2, 3 are at a maximum distance from each other in the plane XY, the end portions 2b2, 3b2 of the second leg portions 2b, 3b of the legs 2, 3 are at a closest distance to each other, and the second leg portions 2b, 2b extend in substantially the same direction in the plane XY along a centre line L extending through the joint connections in the plane XY, a cross-sectional area of the two second leg portions 2b, 3b, including any space in between the second leg portions 2b, 3b, taken perpendicular to the centre line L is at most 4 cm 2. (See FIG. 5)

In one example, the second leg portions have a length of 10 cm, a cross-sectional are of the two second legs (as discussed above) is about 3 cm 2, the angle between the first and second leg portions is 120 degrees, a largest distance between the abutment surfaces of the two second ends of the legs in the plane is 18 cm, and a radius of curvature at at least a portion of the extension of the second leg portion is 10 cm. Such an instrument can be inserted through a small skin incision, such as 3 cm long, and measure a pocket width of up to 18 cm, i.e. a pocket width of about 6 times the width of the incision can be measured.

The second leg portions 2b, 3b of the legs 2, 3 are insertable through the skin incision 10 into the pocket 11 with the legs 2, 3 pivotably connected. Alternatively, the legs 2, 3 are inserted through the incision in a non-connected state and connected once inserted through the skin incision 10. The first leg portions 2a, 3a of the device are arranged at an outside of the incision when the second leg portions 2b, 3b are located in the pocket, see FIG. 1. Once inside the pocket, with the joint connection 2c, 3c at the approximate level of the skin incision, the second leg portions 2b, 3b are caused to move by moving the end portions 2a2, 3a2 of the first leg portions 2a, 3a such that the respective abutment surfaces abuts against an inner wall of the pocket 11.

To measure the inner width of the pocket the device is provided with a distance reader 6, 6′, see FIGS. 2, 3, 5, 6. The distance reader 6, 6′ is arranged to, when the two legs 2, 3 are pivotably connected, give a current closest distance B between the abutment surfaces of the two second end portions 2b2, 3b2 of the second leg portions 2b, 3b of the legs in the plane XY.

When the abutment surfaces abuts against the inner wall of the pocket 11, the measured closest distance B between the abutment surfaces of the two second end portions 2b2, 3b2 corresponds to the inner width D of the pocket 11 at the point of location of the abutment surfaces. To find the widest width D of the pocket 11 it may be necessary to measure the width at more than one position of the device 1 in the pocket 11.

The distance reader 6, 6′ may comprise a digital display (not illustrated) arranged at the device 1 visibly for the user of the device 1 when the second leg portions 2b, 3b of the device are inserted into the pocket, or the distance reader 6, 6′ may be arranged at a position remote from the device 1. Measurement of the distance between the abutment surfaces may comprise use of optical or electronic distance measurement devices.

The distance reader 6, 6′ may be arranged such that a current smallest distance between the abutment surfaces of the two second end portions 2b2, 3b2 of the two second portions 2b, 3b of the legs 2, 3 is derivable from a current distance or angle between the legs 2, 3 at a position between the joint connection 2c, 3c and the end portions 2a2, 3a2 of the first leg portions 2a, 3a of the legs 2, 3. This is illustrated in FIGS. 2, 3, 6.

The distance reader 6 may be constituted by a protractor, see FIGS. 2 and 3, arranged at a position between the joint connection 2c, 3c and the end portions 2a2, 3a2 of the first leg portions 2a, 3a of the legs 2, 3, and wherein the position of one of the first leg portions 2a, 3a at/above the protractor indicates the distance C or angle between the first leg portions 2a, 3a of the legs 2, 3 at the position of the protractor. This distance C or angle corresponding to the current distance B between the abutment surfaces of the end portions 2b2, 3b2 of the second leg portions 2b, 3b of the legs 2, 3 in the plane XY. When the inner width of the pocket 11 has been measured, the device is retracted from the pocket 11 and the skin incision 10. The legs 2, 3 of the device 1 may be disconnected before the device is retracted from the pocket 11. Alternatively, the legs 2, 3 of the device 1 may retracted from the pocket 11 in a connected state.

In FIGS. 5 and 6, an alternative distance reader 6′ is illustrated. It comprises an elongated member 61 rotatably connected to one of the two first leg portions 2a, 3a at a first end of the elongated member. An indicator 62 is fixedly connected to the other of the two first leg portions 2a, 3a, wherein the elongated member 61 and the indicator 62 are arranged such that the indicator 62 is slidable along the elongated member 61 as the first leg portions 2a, 3a of the first and second legs 2, 3 are moved towards and apart from each other. The elongated member 61 is provided with a measurement surface with a scale such that the position of the indicator 62 along the scale correlates with a current distance B between the two abutment surfaces of the end portions 2b2, 3b2 of the second leg portions 2b, 3b of the legs 2, 3.

The elongated member 61 may be provided with an elongated slot 63 extending along the length of the elongated member, along which slot 63 the indicator 61 is allowed to move as the first leg portions 2a, 3a of the first and second legs 2, 3 are moved towards and apart from each other.

The elongated member 61 may be connected to one of the two first leg portions 2a, 3a at a distance from the connection joint 2c, 3c and the indicator 62 may be fixed to the other of the two first leg portions 2a, 3a at substantially the same distance from the connection joint 2c, 3c.

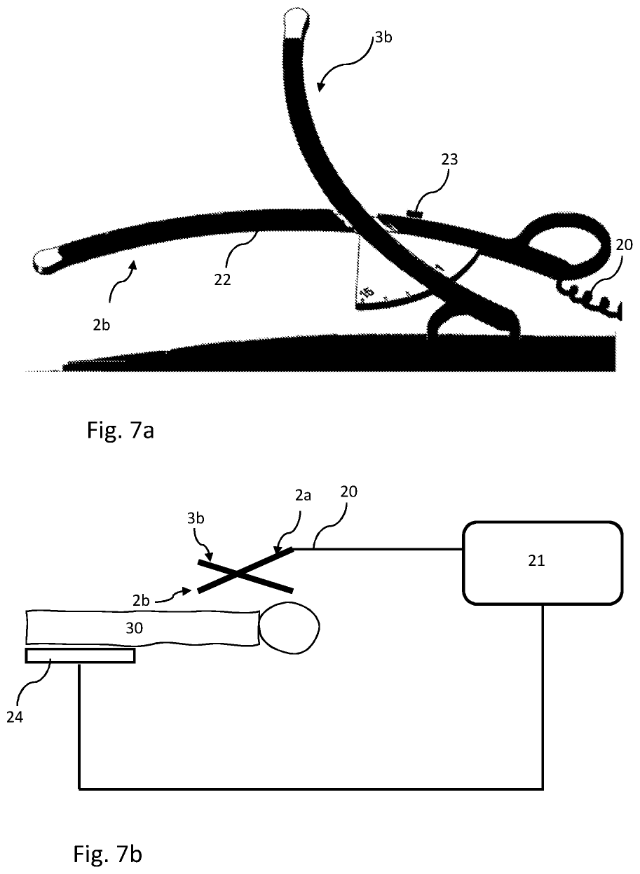

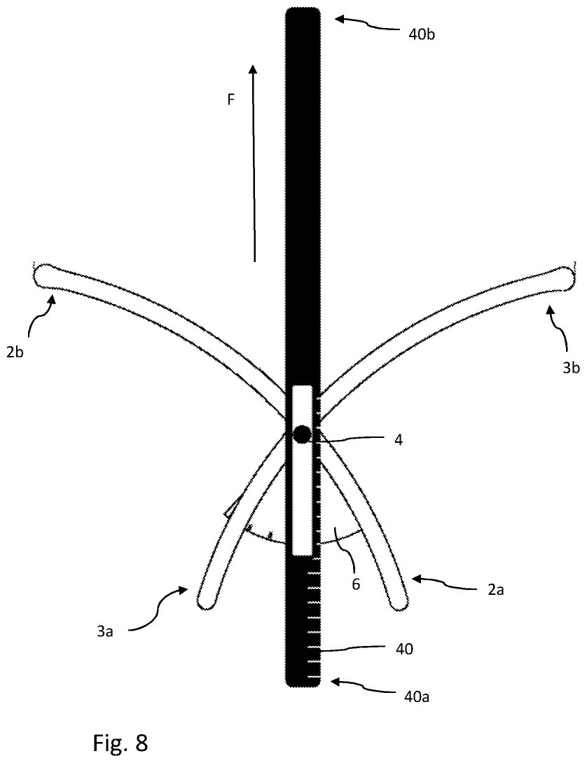

As illustrated in FIG. 7a, the device 1 described above may also comprise cutting means for use when forming the pocket 11. The formation of the pocket 11 may take place before and/or during the measurement of the inner width D of the pocket 11. In FIGS. 7a and 7b is shown that an end portion 2a, 3a of a first leg portion 2a, 3a of the device 1 is provided with a connection 20 to an electrosurgical unit 21. The second end portions 2b, 3b of the legs 2, 3 are arranged as alternating points for a high density current. Apart from the second end portions 2b, 3b of the legs 2, 3 an electrically insulating material 22 covers the legs. A switch controller 23 may be arranged on the device 1 for alternating the high-density current between the two second end portions 2b, 3b. As illustrated in FIG. 7b, a point of low density current 24 is arranged at the subject 30 at a distance from the place of incision. The principle of the cutting means resembling a diathermy knife.

The device 1 may further be provided with an arrangement 40 for measuring a depth of the pocket 11, as illustrated in FIG. 6. This arrangement 40 may comprise a ruler having a first 40a and a second end 40b. The ruler may be slidably connectable to the joint connection 2c, 3c, such that when the abutment surfaces of the end portions 2b2, 3b2 of the two second leg portions 2b, 3b abuts against the inner sides of the pocket 11, the ruler can be pushed into the pocket 11 in a direction F substantially perpendicular to a normal of the abutment surfaces until the second end 40b of the ruler abuts against a wall of the pocket 11. The second end 40b of the ruler may be provided with a curved tip (not shown). The ruler is provided with a distance indication corresponding to the depth of the pocket 11 at the point of measurement.

As shown in FIG. 3, the device 1 may be provided with a light source 50, arranged to provide light into the surgical field inside the pocket 11. The light source 50 may be arranged anywhere on the device 1, and may for example be arranged at or near to the joint connection 2c, 3c. The light source 50 may be powered from a distant power source connected with a cord (not shown), or a battery placed inside or close to the light source 50.

The device 1 is preferably made of a material which is sterilizable and which can withstand sterilization such as one or more of heat, chemicals, irradiation, or high pressure. Examples of such sterilization methods are steam under pressure, dry heat, ultraviolet radiation, gas vapour sterilants, and chlorine dioxide gas.

The material of the device 1 may be a metal alloy, such as stainless steel, titanium alloys, or vanadium alloys, or combinations thereof, or lightweight plastic polymer(s), or a combination of metal alloy(s) and plastic polymer(s). The device may be a reusable device intended for repeated cycles of sterilization and use. Alternatively, the device 1 may be a single-use device.

1. A Device for measuring an inner width of a pocket arranged in a surgical plane deep to skin or underlying tissue of a subject, wherein the device comprises:
two legs, wherein each of the two legs has a first leg portion and a second leg portion, the first leg portion and the second leg portion of each leg is configured to be fixed to each other at a joint connection;
wherein each of the second leg portions being are provided with an abutment surface at a respective end portion configured to abut an inner wall of the pocket;
the two legs are coupled to each other at the joint connection, such that a main direction of extension of the two legs are coupled is in one plane;
wherein the two legs, when coupled, are pivotable relative each other in the plane about an axis perpendicular to the plane, in such a way that when the respective end portion of each of the first leg portions are moved towards each other in the plane, the end portions of the two second leg portions are caused to move apart from each other in the plane, and when the end portions of the two first leg portions are moved apart from each other in the plane, the end portions of the two second leg portions are caused to move towards each other in the plane; and
a distance reader coupled to the device and arranged to give a current distance between the abutment surfaces of the two second end portions of the second leg portions of the two legs in the plane;
wherein an angle between the first leg portion and the second leg portion of each respective leg of the two legs at the joint connection is 90 to 150 degrees.

2. The device of claim 1, wherein when the end portions of the first leg portions of the legs are at a maximum distance from each other in the plane, the end portions of the second leg portions of the legs are at a closest distance to each other and the second leg portions extend in substantially a similar direction in the plane along a centre line extending through the joint connections in the plane, such that a cross-sectional area of the two second leg portions, including any space in between the second leg portions, taken perpendicular to the centre line is at most 4 cm².

3. The device of claim 1, wherein the second leg portions have a length of 4-12 cm.

4. The device of claim 1, wherein a largest closest distance between the abutment surfaces at the end portions of the second leg portions of the legs in the plane is 20 cm.

5. The device of claim 1, wherein at least a portion of the at least one of the second leg portions is arched such that, when the end portions of the two first leg portions of the legs are at a maximum distance from each other in the plane; and
wherein the end portions of the second leg portions of the legs are at a closest distance to each other and the second leg portions extend in a similar direction along a centre line extending through the joint connections in the plane, a radius of curvature of the arched portion of the second leg portion of each leg is located in or extends through an area defined in the plane between the first leg portion and the second leg portion.

6. The device of claim 5, wherein at least a portion of at least one of the second leg portions is arched such that when the end portions of the two second leg portions of the legs are at a closest distance to each other in the plane, and at least a portion of the second leg portion cross at least a portion of the other second leg portion and the centre line in the plane.

7. The device of claim 5, wherein the arched portion is arched such that a radius of curvature is 8-15 cm.

8. The device of claim 1, wherein at least a portion of one of the second leg portions is arched such that there is a curvature in a direction perpendicular to the plane.

9. The device of claim 1, wherein when the end portions of the first leg portions of the legs are at a maximum distance from each other in the plane, and an angle between the first leg portions of the two legs at the joint connection is 130-180 degrees, and an angle between the second leg portions of the two legs at the joint connection is 0-10 degrees.

10. The device of claim 1, wherein the distance reader is arranged such that a current distance between the abutment surfaces of the end portions of the second leg portion of each of the legs is derivable from a current distance or angle between the first leg portions of each of the legs at a position between the joint connection and the end portions of the first leg portions of each of the legs.

11. The device of claim 10, wherein the distance reader is constituted by a protractor arranged at a position between the joint connection and the end portions of the first leg portions of the legs, and wherein the position of one of the first leg portions at or above the protractor indicates the distance or angle between the first leg portions of the legs at the position of the protractor, this distance or angle corresponding to the current distance between the abutment surfaces of the end portions of the second leg portions of the legs in the plane.

12. The device of claim 1, wherein the distance reader further comprises:
an elongated member rotatably connected to one of the two first leg portions at a first end of the elongated member; and
an indicator fixedly connected to the other of the two first leg portions, wherein the elongated member and the indicator are arranged such that the indicator is slidable along the elongated member as the end portions of the first leg portions of the first and second legs are moved towards and apart from each other,
wherein the elongated member is provided with a measurement surface with a scale such that a position of the indicator along the scale correlates with a current distance between the two abutment surfaces of the end portions of the second leg portions of each of the legs.

13. The device of claim 12, wherein the elongated member is provided with an elongated slot extending along at least a portion of a length of the elongated member, and the indicator is allowed to move along the elongated slot as the end portions of the first leg portions of the first and second legs are moved towards and apart from each other.

14. The device of claim 13, wherein the elongated member is connected to one of the two first leg portions at a distance from the joint connection and the indicator is fixed to the other of the two first leg portions at substantially a similar distance from the joint connection.

15. The device of claim 1, wherein the two legs are connected at the joint connection by a mechanical connection, a form-fit connection or a magnetic connection.

16. The device of claim 1, further comprising cutting means configured for forming the pocket.

17. The device of claim 1, further comprising a light source arranged to provide light into the pocket.

18. Method of measuring an inner width of a pocket arranged in a surgical plane deep to the skin or underlying tissue using the device (1) of claim 1, comprising:
inserting the second leg portions of the legs through a skin incision into the pocket;
causing the end portions of the two second leg portions of the device to move apart from each other in the plane such that the respective abutment surfaces of the end portions of the second leg portions abut against an inner wall of the pocket; and
measuring, by means of the distance reader, a current closest distance between the abutment surfaces of the end portions of the second leg portions of the legs, the measured distance corresponding to the inner width of the pocket at a position of the end portions of the second leg portions of the legs of the device in the pocket.

19. The method of claim 18 when using the device, further comprising: forming the pocket before or during measuring the inner width of the pocket.
